(19) United States
(12) Patent Application Publication
(10) Pub. No.: US 2006/0069068 A1
(43) Pub. Date: Mar. 30, 2006

(54) Methods and compositions for the treatment of diseases characterized by pathological calcification

(75) Inventors: E. O. Kajander, Lakeland, FL (US); K. M. Aho, Kuopio (FI); N. Ciftcioglu, Houston, TX (US); B. Millican, Lakeland, FL (US)

Correspondence Address: HOGAN & HARTSON LLP;IP GROUP, COLUMBIA SQUARE, 555 THIRTEENTH STREET, N.W., WASHINGTON, DC, 20004, US

(73) Assignee: Nanobac Pharmaceuticals, Inc.

(21) Appl. No.: 11/102,798

(22) Filed: Apr. 11, 2005

Related U.S. Application Data
(63) Continuation of application No. 10/891,483, filed on Jul. 15, 2004, now abandoned.

(51) Int. Cl. A61K 31/675; A61K 31/195

(52) U.S. Cl. 514/89; 514/566; 514/102

(57) ABSTRACT

Methods and compositions are provided which contains preparations of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents and which may be used for treating and or reducing pathological calcifications, the growth of Nanobacterium and calcification-induced diseases including, but not limited to, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) in humans and animals. The method comprises administering the various classes of compositions of the present invention, which together effectively inhibit or treat the development of calcifications in vivo.

### BACKGROUND OF THE INVENTION

1. Field of the Invention

The invention relates to therapeutic methods and compositions for the treatment of calcification and/or plaque-based conditions associated with nanobacterial infection, and more particularly to therapeutic compositions and methods for treating and/or preventing the growth of Nanobacterium and other calcifications by administering preparations of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents that may include amino acids enzyme systems antioxidants and natural anti-inflamatory compositions.

2. Discussion of the Related Art

The formation of discrete and organized inorganic crystalline structures within macromolecular extracellular matrices is a widespread biological phenomenon generally referred to as biomineralization. One example of biomineralization is the formation of calcium phosphate. When calcium phosphate is deposited in tissue, it is known as calcification. Mammalian bone and dental enamel are examples of calcification.

The mechanism of normal calcification is poorly understood. Yet, an average human has 700 g of calcium phosphate in bones and teeth. Also, all cells in the body require intracellular calcium, which allows mammalian cells to regulate multiplication, repair systems, signal transduction and metabolism. Intracellular calcium is the most important second messenger in mammalian cells, and blocking that action would seriously harm cells and tissue functions.

Pathological calcification is not the healthy process that builds bones and teeth, but instead it is found in disease. Most pathologic calcifications contain mixtures of nonacidic carbonate-substituted hydroxyapatite and octacalcium phosphate, referred to as basic calcium phosphate (BCP). Unfortunately, there is no clinically useful definitive assay for BCP crystals. Clumped crystals can be identified only with transmission electron microscopy. They are not birefringent under polarized light microscopy. On x-ray, they may be visible as periarticular cloudlike opacities.

Pathological calcification is found in a variety of diseases. While the cause of pathological calcification remains unknown, researchers have observed a common link in each of these diseases—that is, the presence of a very small mineral-associated bacteria-like life form called Nanobacteria. The term “Nanobacteria” describes the scientific genus, a species of which is named Nanobacterium sanguineum. Nanobacteria (“NB”) are approximately 50-200 nanometers in size and are currently the smallest known self-replicating agents and must be separated from common bacteria (Eubacteria and Archaebacteria).

Nanobacteria induce calcium phosphate mineralization under physiologic, or lower concentrations of Ca²⁺ and PO₄— thus acting as active nidi for crystallization, whereas other nidi, such as apatite particles, are passive and cannot function under non-saturating calcium-phosphate concentrations. Nanobacteria form carbonate apatite crystals, but other calcium phosphates may be present, including amorphous calcium phosphate, brushite and hydroxyapatite. Nanobacteria are nano-sized in that they are from 20-200 nanometers in size and are described as the smallest known self-replicating bacteria.

Nanobacteria are active nidi for calcium phosphate crystallization in that that they actively produce the calcium phosphate minerals, BCP, or carbonate apatite. Nanobacteria produce calcification by building calcium-phosphate mineral deposits or “envelopes” around each nanobacterial cell. Nanobacterium secretes a calcific biofilm around itself that protects the Nanobacteria and allows for multiple Nanobacteria to connect, collaborate and apparently form together as a unit or colony. This calcific biofilm also allows the Nanobacterial to expand, contract and move. In addition to being active nidi in pathological calcifications, Nanobacteria contain endotoxin and possibly more mediators of inflammation. Endotoxin is known to be the most powerful mediator of inflammation. Thus, Nanobacteria activate not only calcification, but also inflammation.

The biofilm-phase appears to be present when Nanobacteria are chemically attacked, physiologically stressed, environmentally attacked or when they are working together or reproducing. During the biofilm-phase, when Nanobacteria secretes the calcium-phosphate mineral, the Nanobacteria is most harmful because it forms calcified plaques. The body has only little action possibilities against biofilms, especially calcific plaques, which is evidenced by the fact that calcification remain inside fibrous capsules for years, and the body cannot eliminate them.

Many of the medical markers of inflammation, such as C-reactive protein, are found to be elevated in response to the endotoxin in the Nanobacteria biofilm. Nanobacteria has been shown to pathologically activate cells, immunological responses, thrombotic cascades, and neuro-muscular systems. The calcified plaques caused by Nanobacteria activates a thrombic cascade. Nanobacteria have calcium phosphate coat and thus bind prothrombin and prothrombinase complex. This leads to activation of thrombotic cascade because many proteins in the blood coagulation cascade have high affinity to calcium phosphate. Also, the calcific biofilm that is secreted by the Nanobacteria is a potent endotoxin and causes inflammation and swelling, causing the surrounding tissue to respond by releasing cytokines, interleukins, leukocytes, mast cells, collagenase, matrix metalloproteinases and other immune-responsive reactions.

Nanobacteria may also form the calcific biofilms and replicate under blood/serum conditions. Nanobacteria are the only calcium-phosphate mineral containing particles isolated from human and cow blood that are cytotoxic in vitro and in vivo. Human and bovine Nanobacteria grow similarly, share the same surface antigens and various other features. They both produce biomineralization. Most biologicals and vaccines are made from fetal bovine serum, which raises a significant safety concern. Furthermore, Nanobacteria has been found to be a contaminant on previously-assumed-to-be sterile medical products, such as tissue, blood and bovine serum.

In addition to the pathological calcification caused by Nanobacteria, trauma, stress, surgery and/or biological implants are associated with pathological calcification and the presence of Nanobacteria. Nanobacteria can adhere practically to any surface material and their presence enhances the formation of mixed bacterial biofilm. Foreign objects are known to calcify in the human body. In particular, biological implants (e.g. prosthesis) are vulnerable to undesired calcification. Bioprosthetic devises in which calcification is a serious problem include, but are not limited to, heart bioprotheses, homografts/allographs (human cadaver), autografts, mechanical bioprotheses and implants, particularly those made using polyetherurethaneurea and polyetherurethane, silicone implants (including breast implants) and other synthetic materials.

In a similar way, infectious and parasitic agents can be walled by calcification. Furthermore, necrotic tissues can calcify. All these forms of pathological calcification involve local elevation in calcium and phosphate concentrations. Similarly, intracellular calcium vesicles are released and can interact with phosphate released from necrotic tissue. Phosphate is normally present intracellularly at about 100 mM levels. Although most of it is bound to other molecules, the intracellular pool of phosphate is huge, 100-fold larger than phosphate levels in serum (around 1 mM). Phosphate in nucleotides (about 10 mM pool) is released in minutes after start of anoxia. Alkaline phosphatase and other enzymes can release phosphate from nucleic acids, proteins, alpha gycerophosphate, and phospholipids slowly resulting in huge load of phosphate, because there is no circulation in necrotic tissue. Thus especially phosphate contributes to huge ion product supersaturation with respect to apatite formation. This is to the contrary of pathological calcification found in apparently normal tissues, cardiac valves, arteries and early cancer, where there is no evidence for necrotic lesions, and nanobacteria are the only plausible explanation for pathological calcification.

Nanobacteria induced pathological calcification is resistant to systemic therapy. Nanobacteria are extremeophiles and probably the most highly resistant of all bacteria to destruction. There are presently no known naturally occurring substances that can eliminate the Nanobacteria. Additionally, Nanobacteria cannot be killed using most antibiotics, such as Penicillin, Cephalosporins, or Macrolides. Nanobacteria are also tolerant to very high heat, freezing, dehydration and Gamma Irradiation. Studies on gall stones, kidney stones, pancreatic stones and dental stones have shown that calcium phosphate stones, such as those involving Nanobacteria, cannot be dissolved with any previously known systemic oral therapy. As a comparison, kidney stones consisting of urate can be treated with allopurinol. Struvite kidney stones can be dissolved by urine acidification. Gall-stones contain cholesterol, bilirubin, bile acids and calcium phosphate can be dissolved using bile salts and bile salt derivatives, terpenes and other organic solvents. However, gall-stones with high calcium phosphate content cannot be effectively dissolved.

The ability to study Nanobacteria has been difficult. Many of the chemicals used to stain cell walls or other components of traditional bacterial fail to bind to Nanobacteria. Also, Nanobacteria do not thrive on agar, the medium used to grow most bacteria. As such, the ability to culture Nanobacteria and to develop Nanobacterial antibodies has been difficult. Commonly assigned U.S. Pat. No. 5,135,851, incorporated herein by reference, describes a culture method for Nanobacteria.

Nanobacteria cannot be grown on standard media for bacteria, and thus they escape detection when using standard culture methods. The detection of the extremely small unidentified bacteria is hampered by their size, which, e.g., in commercial cell culture isolates, is smaller than 0.5 micro-meters. Thus, their detection via light microscopy is possible only with the best microscopes having maximum resolution. Tissue culture laboratories are seldom equipped with such microscopes. Further, these bacteria are difficult to collect since centrifugation is difficult. They are also readily lost since they do not adhere to glass in standard fixation treatments, and they cannot be stained with common bacteriological stains. The growth requirements of species of bacteria of the genus Nanobacterium are quite similar. The growth requirements can be met using standard tissue culture media. This is likely because these bacteria are adapted for living inside the mammalian body.

The ability to detect Nanobacteria is also very difficult. As noted above, Nanobacteria are extremely small, approximately 1/1,000 the size of most other bacteria. Nanobacteria are also very slow growing agents, reproducing every 3 days. Most other forms of bacteria reproduce in minutes or in hours. Also, Nanobacteria are pleomorphic in that they have varying forms or shapes during their life cycle and can change appearance and form during growth and development. Because of their extremely small size, slow growth rate, and pleomorphism, Nanobacteria often avoid detection. Commonly assigned U.S. Pat. No. 5,135,851, incorporated herein by reference, describes a method for detecting Nanobacteria.

Pathological calcification induced by Nanobacteria is common. Nanobacteria has been observed to be the cause of pathological calcification found in a wide range of diseases. Nanobacteria induced pathological calcification is implicated to be either the cause or instrumental component of most all degenerative disease processes. These include Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease).

Nanobacteria induced pathological calcification has been shown to cause these many disease states through the biochemical and pathophysiological mechanisms for calcium pathogenesis. Biochemically, calcium is the most important intracellular second messenger in mammalian cells. It is a very convenient messenger, because it is always present in the extra-cellular medium at high levels, about 2.5 mM, of which about half is free ionized calcium. Mammalian cells have calcium channels that can be opened and closed. If they are opened, there is influx of calcium which then binds to calcium sensitive metabolic switches that activate the cell. Activation is rapidly stopped by pumping calcium back to extra-cellular space or to intracellular vesicles.

Researchers have shown that changes in the cytoplasmic calcium concentration ([Ca(2+)](i)) regulate a wide variety of cellular processes. Increased [Ca(2+)](i) can induce hormone-independent survival and proliferation, as well as evoke apoptosis in human myelo-erythroid GM-CSF/IL-3 dependent leukemia cells (TF-1). Cellular responses induced by elevated [Ca(2+)] depended on the duration and amplitude of the calcium signal. Moderate or high, but transient, elevation of [Ca(2+)](i) causes a transient, biphasic activation of ERK1/2 and protected cells from hormone withdrawal-induced apoptosis. In contrast, high and long-lasting elevation of [Ca(2+)](i) leads to sustained activation of the ERK1/2 kinases and apoptosis of TF-1 cells. Data suggests that a time-dependent action of the MAPK pathway works as a decision-point between cell proliferation and apoptosis.

Other researchers have obtained similar results with various calcium phosphate phases. Basic calcium phosphate crystals (BCP) cause abnormal Ca²⁺ signalling in cells. BCP crystals have growth stimulating effects on many cells, as exampled by fibroblast-like synoviocytes, chondrocytes, human breast cancer cells and fibroblasts. Ability to promote mitogenesis is of great importance in malignant cells, where apatite particles thus promote the growth of a tumour. Stimulation of mitogenesis in non-transformed cells can lead to hyperplasia and benign tumours. For example, PKD, a genetic disorder that strikes as many as 1 in 500 people, is characterized by the formation of large cysts caused by uncontrolled kidney epithelial cell division driven by calcium.

Addition of BCP crystals to cultured mammalian cells results in rapid rise in intracellular Ca²⁺ entering via Ca²⁺ channels. Dissolution of phagocytosed BCP crystals causes a second, longer-lasting rise in intracellular Ca²⁺. Crystal-dissolution-mobilized Ca²⁺ diffuses into the nucleus through nuclear pores and enchances c-fos mRNA expression. BCP crystals induces c-fos via calcium-dependent Protein Kinase C (PKC) pathway and calcium-independent mitogen-activate protein kinase (p44/42 MAPK) pathway mediated by PKCμ. c-fos induction in the nucleus leads to expression of matrix metalloproteinases (MMP) 1 and 3 and mitogenesis via AP-1. BCP has also been shown to induce mRNA of matrix metalloprotein-8 in human fibroblasts in vitro. Thus, calcium exerts a multi-targeted role in cell activation and function that is linked to atherosclerosis, cancer and tissue loss and impaired function.

In addition to the biochemical mechanisms described above, pathophysiological mechanisms for calcium pathogenesis likewise show that Nanobacteria induced pathological calcification causes these many disease states. For example, as mentioned previously, Nanobacteria have calcium phosphate coat and thus bind prothrombin and prothrombinase complexes, which can lead into activation of thrombotic cascade. Apatite has been used for purifying commercial prothrombinase complex (containing prothrombin=factor 2, factor 7, factor 9, factor 10), which partially activates the complex even during the purification taking place at a low temperature. Active thrombin is released by activated factor 10 (Calcium activates it) because this has no gamma carboxylated glutamates. Thrombin then splits fibrinogen into fibrin forming the thrombus, either alone (white thrombi) or with platelets and eryhtrocytes (red thrombi). Massive thrombotic events have been found in laboratory animals injected intravenously with Nanobacteria.

Nanobacteria induced pathological calcification has been shown to be linked to autoimmune responses. Autoantibodies have been observed in nanobacteria-injected mice. Nanobacteria, which has a calcium phosphate coat, avidly binds from its surroundings proteins and DNA, and can thus be transported into a novel host and exposed to immune system or expressed in the host cells. This means that the foreign DNA may start autoimmune reaction by expressing a foreign protein, may transform cells after being incorporated into nucleus or may result in DNA immunization. This mechanism is thus liable to pathogenicity with respect to transformation into cancer cells and autoimmune diseases.

Nanobacteria induced pathological calcification has also been linked to tissue calcification. Scleroderma, which involves massive calcification of the skin and has a very poor prognosis. Juvenile dermatomyositis involves skin and muscle, is considered to be a vaccination complication with a frequency of 1 out of a million and has also very poor prognosis. Rheumatoid arthritis patients often develop massive soft tissue calcification around areas of bone ulceration, that severely compromises the patient's ability to use his/her affected joints. Arteritis nodosa involves inflammation and thrombosis of arteries. It can resemble calciphylaxis, where there is massive calcification of arteries and thrombotic necrotic lesions. Autoimmune polyglandular endocrinopathy syndrome may involve Grave's disease, autoimmune hypothyreoidism, hypopituitarism, type 1 diabetes mellitus, autoimmune Addison's disease and hypoparathyreoidism. It involves tissue destruction, cyst formation and tissue calcification.

Nanobacteria induced pathological calcification has also been linked to cancer and other diseases related to altered cell functions & cell transformation. These are examples of diseases caused by an overproduction of growth factor. Because calcific crystals can bypass the growth factor, such diseases could be aggravated by the simple presence of tissue calcifications such as those caused by Nanobacteria. The Nanobacterial calcium phosphate coat can release calcium on contact into mammalian cell or when nanobacteria are internalized by cells. This can elevate intracellular calcium levels. ([Ca(2+)](i)) regulate a wide variety of cellular processes. Studies have demonstrated that increased [Ca(2+)](i) is able to induce hormone-independent survival and proliferation, as well as to evoke apoptosis in human myelo-erythroid GM-CSF/IL-3 dependent leukemia cells (TF-1). Thus calcium exerts a multi-targeted pathology in cell activation and function. DNA-calcium phosphate precipitates are taken up by cells, which is exploited in transfection of mammalian cells. DNA transfection leads to altered cell function, protein and DNA expression, and may result in transformation into malignant cells. It is of interest in this respect that benign cysts can undergo malignant transformation with concomitant appearance of calcification. For example, embryologic remnants of thyroid tissue often line the thyroglossal duct tract and may commonly become cystic. Calcification in such a cyst is thought to be a specific marker for malignancy, which may develop in 1% of thyroglossal duct cysts. Also meningeomas, breast cancers, ovarian and prostate cancers have calcification very commonly. The extent of calcifications appears to have significant prognostic value in metastasis potential at least in breast cancer. Studies have assessed the presence of invasion in breast cancer with microcalcifications and have investigated the correlation between the area of calcification, morphology of calcification on mammography, histological subtype of intraductal carcinoma (comedo or non-comedo) and frequency of invasion, and lymph node metastasis. Invasion has been observed in 33 of 157 pts (21%). The risk of invasion was 10% within 10 mm of punctate-round and amorphous type microcalcifications, and 37% at more than 11 mm of pleomorphic, linear-branching microcalcifications. The specific role for calcification in cancer is obvious even from the metastatic cancers: metastatic cancers often retain their original calcification patterns. Peripheral “egg-shell” calcifications have also been demonstrated in renal metastasis from papillary thyroid carcinoma. Further, completely calcified monofocal calcification have been demonstrated in renal metastasis from osteosarcoma. Thus, calcified renal metastases are rare lesions related to specific oncotypes. Diagnosis is based on a history of specific oncotypes (papillary and mucin-secreting carcinomas, osteosarcoma and chondrosarcoma). Thus, measures reducing tissue calcification can influence malignant transformation and metastasis potential of cancer, because tissue calcification can act as uncontrolled growth stimulant independent from growth factor(s). Malignant transformation is a stepwise phenomenon involving several mutations, the probability of which is dependent on cell divisions (no divisions=probability is zero). This process can be supported by calcification stimulating cell divisions. Certain malignancies retain their calcification even at the metastatic stage, and thus anticalcification therapy may reduce their metastatic potential.

Nanobacteria induced pathological calcification has also been linked to altered membrane lipids & lipid permeation. Altered function can involve cell membrane permeability increase observed in atheromas contributing to pathology of cholesterol and other lipids. Calcium stimulates uptake of cholesterol and other lipids to macrophages and macrophages' oxidation of lipids, thus activating soft plaque formation in atheromas. Endothelial permeability is also increased by calcium. Because Nanobacteria are internalized in lysosomal membrane vesicles in mammalian cells, they may carry lysosomal myeloperoxidase on their apatite coat when the cells die or when Nanobacteria escape. Extracellular myeloperoxidase activity is a well-known risk factor for acute myocardial infarction (MI).

Nanobacteria induced pathological calcification is also liked to apoptosis & loss of tissue structure & function. Apoptosis can be involved in many diseases including chronic heart failure, loss of ciliated cells in Polycystic Kidney Disease and in airway pathology, chronic obstructive pulmonary disease (COPD), and loss of function like blindness and loss of hearing due to the nerve cell degeneration, loss of neuronal function resulting in dementias, such as Alzheimer's disease, or degenerations, such as amyotrophic lateral sclerosis and various neuropathies including HIV and diabetic neuropathy. High and long-lasting elevation of [Ca(2+)](i) leads to sustained activation of the ERK1/2 kinases and apoptosis of TF-1 cells, suggesting that a time-dependent action of the MAPK pathway works as a decision-point between cell proliferation and apoptosis. Also, hydroxyapatite ultrafine powder has been shown to cause DNA damage in W-256 sarcoma cells and in lesser amounts in rat lymphocytes. Researchers have addressed the biochemical mechanism of cyst formation in polycystic kidney disease (PKD) where increased apoptotic cell death is a pathognomic finding in the kidneys developing cysts. They showed that mechanical movement of the kidney cilia causes a massive influx of calcium into the kidney cells, and that the calcium almost certainly travels through the polycystin ion channels on the cilia. These findings suggest the kidney cilia probably act as mechanical sensors that respond to fluid flow in the kidney by bending, and admitting calcium through the polycystin channels on their surface in the process. When kidney cilia are mechanically bent, and calcium flows into the cells, a piece of the calcium channel is clipped and goes to the nucleus where it affects transcriptional regulation from genes. This provides a direct link between the mechano-sensory calcium channels on the ciliary membrane and nuclear regulation of the cell. Intrestingly, nanobacteria have been found in all cysts of PKD patients studied.

Nanobacteria induced pathological calcification has also been linked to serum levels of cholesterol, bile acids, bilirubin and other lipids. Enriching endoplasmic reticulum membranes with unesterified cholesterol (FC) to a level that occurs in the ER of FC-loaded macrophages is capable of markedly increasing the order of ER membrane lipids, and this increase in lipid order is strongly correlated with inhibition of sarcoplasmic-endoplasmic reticulum calcium ATPase-2b SERCA2b, which is the calcium pump responsible for maintaining ER calcium stores in macrophages. It is speculated that SERCA2b, a protein with eleven membrane-spanning regions that undergoes multiple conformational changes during its calcium pumping cycle, loses function due to decreased conformational freedom in FC-ordered membranes. This biophysical model may underlie the critical connection between excess cholesterol, UPR induction, macrophage death, and plaque destabilization in advanced atherosclerosis. Similarly, gall stones have an inorganic framework of calcium and phosphate (CaP). This mineral framework is of importance for growth of gall stone. Calcium binds bilirubin, bile acids and can absorb cholesterol.

Nanobacteria induced pathological calcification has also been linked to atherosclerosis. Atherosclerosis is an inflammatory disease characterized by injury or infection of the vascular endothelium resulting in the formation of atheromas and pathological calcification. Inflammatory cascade responses within individual atheromas result in the synthesis of a fibro-lipid matrix synthesis and the degradation/absorption of soft plaques. The rate of plaque synthesis-resorption is dependent upon the degree and/or stage of inflammatory activity within atheroma. Mature atheromas, for example, contain pathological calcification deposits that have been observed to increase at an annual rate of 24-82%. Although pathological calcification deposits are a hallmark of atherosclerosis, the precise mechanism of such calcium precipitation has remained elusive. It has been widely speculated, however, that Nanobacteria play a critical role in the pathological calcification processes associated with atherosclerosis. In particular, Nanobacteria have been detected atherosclerotic plaques, calcified carotid arteries, aortic aneurysms and cardiac valves. Furthermore, Nanobacteria particles morphologically and functionally resemble the calcifiable vesicles, are capable of active calcium phosphate precipitation under suitable nutrient conditions and have previously been isolated from atherosclerotic aorta.

Because Nanobacteria and prostheses cause and or increase pathological calcification, and because pathological calcification is increasingly liked with numerous diseases, it would be advantageous to provide unique treatments and protocols which can be used to inhibit and/or prevent calcification and calcification-induced diseases in vivo and to inhibit and/or prevent the growth of Nanobacteria in vivo.

### SUMMARY OF THE INVENTION

The invention provides a methodology as well as compositions for treating pathological calcifications, pathological calicification-induced diseases, and in particular, for treating and/or preventing the growth of Nanobacterium. The invention further provides for a protocol for treating and/or reducing calcification and calcification-induced diseases that includes the administration of preparations of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents.

In accordance with embodiments of the invention, the calcium chelators may include one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

In accordance with the invention, the biophosphonates may include one or more of alendronate, clodronate, ibandronate, incadronate, neridronate, palmidronate, risedronate, tiludronate, zoledronate, etidronate, oxidronate, and pharmaceutically acceptable salts thereof.

In accordance with the invention, the antibiotics may include one or more of beta-lactam antibiotics, aminoglycoside antibiotics, tetracyclines, trimethoprim and sulpha-trimethoprim combinations, nitrofurantoin, and pharmaceutically acceptable salts thereof.

In accordance with the invention, the methods and compositions may also include antimicrobial agents, or anti-metabolites, or cytostatic agents against Nanobacteria.

In accordance with the invention, the calcium ATPase and pyrophosphatase pump inhibitors may include one or more of bisphosphonates, vitamin C, vanadate, fluoride, N-ethylmaleimide, N,N-dicyclohexylcarbodiimide, imidodiphosphate, bafilomycin A, calcimycin, or other antibiotics.

In accordance with the invention, the calcium phosphate-crystal dissolving agents may include, in addition to the calcium chelators referenced above, one or more of citrate, lactate, bisphophonates, or other organic and inorganic acidic compounds, including sodium and potassium salts, magnesium citrate, phosphocitrate and other complexes of citrate.

In accordance with the invention, the agent effective against calcium phosphate-crystal nucleation and crystal growth may include one or more of pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom.

In accordance with the invention, the supporting agents may include bile acids and their derivatives and/or terpenes and other organic solvents to dissolve cholesterol and bilirubin, anti-lipemic drugs including statins and others, anti-platelet agents, anti-blood clotting agents, non-steroidal anti-inflammatory drugs, immunomodulators including statins, or a combination of amino acids, vitamins, antioxidants, anti cell death agents, matrix metalloproteinase inhibitors, enzyme systems and inhibitors of calcium-mediated mixed bacterial biofilm formation, such as antibiotics, fluoride, bisphosphonates, calcium chelators and citrate compounds and other calcium-sequestering acids. These supplements enhance the efficacy of the other agents described above.

### BRIEF DESCRIPTION OF THE FIGURES

FIGS. 1 and 2 depict the biofilm formation of Nanobacteria.

FIG. 3 depicts Nanobacteria entering to E. coli cells.

FIG. 4 depicts agrobacteria tumefaciens-nanobacteria mixed biofilm.

FIG. 5 depicts the effect of some chelating agents, apatite crystal poisons and mixed compounds on the growth of nanobacteria as measured by turbidometry, after eight-day growth period.

FIG. 6 depicts agents inhibiting or activating known vacuolar H⁺-PPase and Ca⁺-ATPase pumps.

### DETAILED DESCRIPTION OF THE PREFERRED EMBODIMENTS

The invention provides for therapeutic compositions and methods for treating and/or preventing the growth of Nanobacterium and pathological calcifications by administering preparations of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents, and for treating and/or preventing calcification-induced diseases including, but not limited to, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease).

The methods involve administering to a patient a therapeutically effective amount of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents. In view of the foregoing, the methods of this invention are particularly applicable where the patient is at risk for or has Nanobacterial infection and where the patient will have or has had surgery and/or biological implants.

The composition of the present invention comprises a mixture of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents as the primary therapeutic agents to be administered for the purpose of reducing and/or preventing pathological calcifications, Nanobacterium, and preventing calcification-induced diseases including, but not limited to, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) in an individual in need thereof.

As discussed above, Nanobacteria produces biomineralization by forming a calcific biofilm and calcium phosphate crystals. The mineral coating constitutes a part of the cell wall essential for survival strategy of the organism. Nanobacteria uses the calcific biofilm to catalyze its metabolic processes and to provide it with structural support.

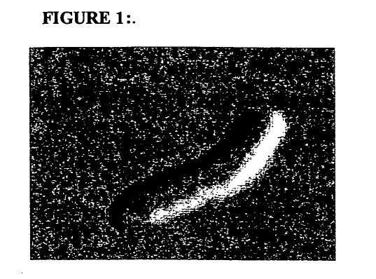

FIGS. 1 through 4 depict biofilm formation, structure and Nanobacteria-bacteria interactions. FIG. 1 depicts a singular and ordinary E. coli with an oval shape (×100). Co-culture of Acrobacterium tumefaciens and E. coli DH5 alpha with Nanobacteria showed enchanced biofilm formation with Acrobacterium tumefaciens and tight adherence and internalization to E. coli DH5 alpha. As shown in FIG. 1, nanobacteria link A. tumefaciens cells in a biofilm. The interaction is likely to be Calcium-mediated as nanobacteria particles contain calcium containing hydroxyapatite envelopes. Calcium is a known mediator of bacteria biofilm formation.

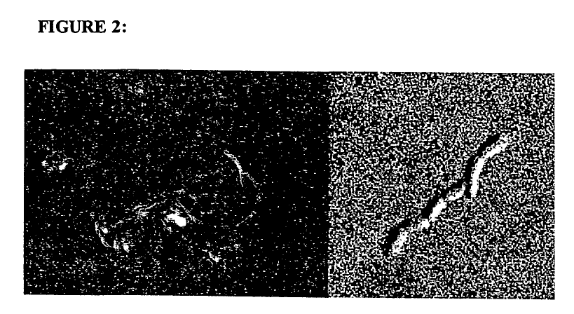

FIG. 2 depicts biofilm between E. Coli and Nanobacteria (×100). The shape of E. coli was elongated when exposed to Nanobacteria strain NBCS. Nanobacteria were adherent to E. coli cells and possibly internalized into E. coli.

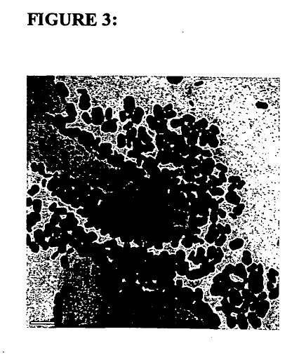

FIG. 3 depicts Nanobacteria adhered and entering to E. coli DH5-alpha cells (TEM picture 30000× magnification).

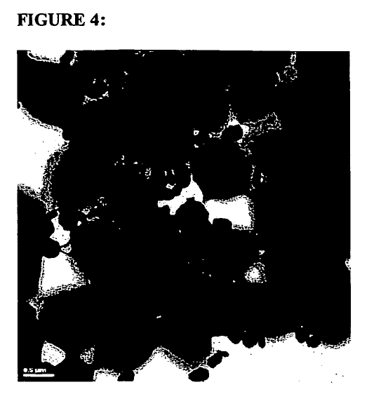

FIG. 4 depicts agrobacteria tumefaciens-nanobacteria mixed biofilm with negative staining, omitting Uranyl Acetate. (TEM picture 25000× magnification). These results were obtained using light microscopy and point out that Acrobacterium tumefaciens grow slower when Nanobacteria are present. Nanobacteria induce biofilm formation of A. tumefaciens. Nanobacteria seemed to hinder the metabolism of studied bacteria and leading slowly toward structural changes or apoptosis. One reason might be adherence on the plasma membrane surface which occurred in the case of Acrobacterium tumefaciens (See FIG. 1). E. coli Nanobacteria may have an influence on the functions of phosphatase transporters and thereby on the translocation of the amino-terminal signal peptide to the periplasmic side of the cytoplasmic membrane.

A calcium chelator that is targeted to the calcific biofilm may be useful for the treatment of pathological calcifications, Nanobacterium, and calcification-induced diseases. The calcium chelators currently available for use in the present invention and the associated daily recommended dosage include Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof. The dose of these medicines will be variable for different patients.

Similarly, calcium phosphate-crystal dissolving agents may be useful for the treatment of pathological calcifications, Nanobacterium, and calcification-induced diseases. The calcium phosphate-crystal dissolving agents currently available for use in the present invention and the associated daily recommended dosage include, in addition to calcium chelators, a variety of citrate, lactate and other organic and inorganic acidic compounds, such as sodium and potassium salts, magnesium citrate, phosphocitrate and other complexes of citrate, and some bisphosphonates. The dose of these medicines will be variable for different patients.

Bisphosphonates, may also be useful to block calcium and phosphate accumulation, and bisphosphonates may also be effective against calcium phosphate-crystal nucleation and crystal growth. Bisphosphonates are characterized pharmacologically by their ability to inhibit bone resorption, whereas, pharmacokinetically, they are classified by their similarity in absorption, distribution, and elimination. Bisphosphonates have a P—C—P bond instead of the P—O—P bond of inorganic pyrophosphate that makes them resistant to enzymatic degradation and gives them a high affinity for hydoxyapatite. They are potent blockers of osteoclasic bone resorption and have been successfully used to treat metabolic bone diseases that involve increased bone resorption. Bisphosphonates also inhibit bone mineralization and soft tissue calcification.

Bisphosphonates suitable for use in the present invention include, but are not limited to, alendronate, clodronate, ibandronate, incadronate, neridronate, palmidronate, risedronate, tiludronate, zoledronate, etidronate, oxidronate, and pharmaceutically acceptable salts thereof. It is possible to synthesize a variety of bisphosphonates by substituting the hydrogen on the carbon atom. The dose of these medicines will be variable for different patients.

Other agents that may also be useful to block calcium and phosphate accumulation, and may thereby be useful for the treatment of pathological calcifications, Nanobacterium, and calcification-induced diseases include calcium ATPase and pyrophosphatase pump inhibitors including, in addition to bisphosphonates mentioned above, vitamin C, vanadate, fluoride, N-ethylmaleimide, N,N-dicyclohexyl carbodiimide, imidodiphosphate, bafilomycin A or calcimycin or some other antibiotics. The dose of these medicines will be variable for different patients.

Other agents that may also be effective against calcium phosphate-crystal nucleation and crystal growth and may thereby be useful for the treatment of pathological calcifications, Nanobacterium, and calcification-induced diseases include, in addition to bisphosphonates mentioned above, pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom.

Antibiotics, anti-microbial agents, anti-metabolites and cytostatic agents reduce and/or prevent pathological calcifications, Nanobacterium, and calcification-induced diseases by an independent mechanism of action. Unlike some of the other compounds mentioned above, antibiotics, anti-microbial agents, anti-metabolites and cytostatic agents inhibit enzymatic reactions that are vital for cells to reproduce. Thus, the present invention also contemplates the use of antibiotics, anti-microbial agents, anti-metabolites and cytostatic agents to reduce and/or prevent pathological calcifications, Nanobacterium, and calcification-induced diseases.

Moreover, the various classes of compounds of the present invention (e.g. calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents) are expected to have a synergistic effect on reducing and/or preventing pathological calcifications, Nanobacterium, and calcification-induced diseases.

Commonly assigned U.S. Pat. No. 6,706,290, which is incorporated herein by reference in its entirety discloses a method for preventing the development of calcifications in vivo in a patient in need of such treatment comprising administering an antibiotic. The antibiotics currently available for use in the present invention and the associated recommended daily dosage include, but are not limited to, the group consisting of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof. Suitable beta-lactam antibiotics for use in the present invention include, but are not limited to, penicillin, phenethicillin, ampicillin, aziocillin, bacmpicillin, carbenicillin, cylclacillin, mezlocillin, piperacillin, epicillin, hetacillin, cloxacillin, dicloxacillin, methicillin, nafcillin, oxacillin, and pharmaceutically acceptable salts thereof. Suitable aminoglycoside antibiotics for use in the present invention include, but are not limited to, streptomycin, kanamycin, gentamycin, amikacin, neomycin, pardomycin, tobramycin, viomycin, and pharmaceutically acceptable salts thereof. Suitable tetracyclines include, but are not limited to, tetracycline, chlortetracycline, demeclocycline, doxycycline, methacycline, oxytetracycline, rolitetracycline, minocycline, sancycline and pharmaceutically acceptable salts thereof. The dose of these medicines will be variable for different patients.

The present invention also provides for a combination of supportive agents, also referred to herein as supplements or neutroceutical powder, including bile acid derivatives, terpenes, organic solvents, anti-lipemic drugs, statins, anti-platelet agents, anti-blood clotting agents, non-steroidal anti-inflammatory drugs, immunomodulators, amino acids, vitamins, antioxidants, anti cell death agents, matrix metalloproteinase inhibitors, enzyme systems, antibiotics, fluoride, bisphosphonates, calcium chelators, citrate compounds and calcium-sequestering acids.

These supportive agents dissolve non-mineral components of the stone or calcification, and prevent calcium-mediated mixed bacterial biofilm formation. They also protect against blood clotting and thrombosis induced by exposed calcium surface. The supportive agents also improve drug penetration and tissue blood flow, and prevent tissue destruction while improving tissue remodeling and tissue healing, or controlling inflammation and immune response.

In one embodiment, the supporting agents may include a combination of Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain. The nutraceutical powder can also include other ingredients and materials as described herein.

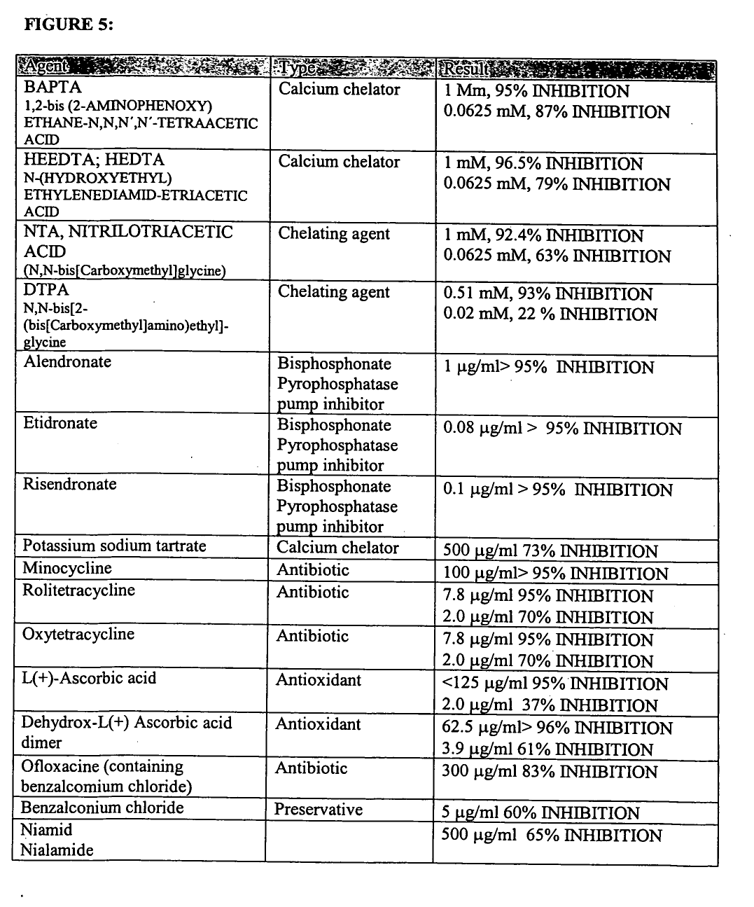

The effect of some chelating agents, apatite crystal poisons and mixed compounds on the growth of nanobacteria as measured by turbidometry, after 8 day growth period is shown in FIG. 5. In the assay produre. Nanobacteria were suspended in 20% FBS-90% DMEM for turbidity value 4-8 ntu. Chemicals were dissoluted and siluted in DMEM, and sterile filtered. Nanobacteria suspension was added 1 part and chemical dilution 1 part for five Ø3 cm dishes. Two dishes were used for baseline turbidity value measurement. Three dishes were transferred to cell culture incubator +37° C. 95% air-5% CO₂ for 8 days. At day 8, cultures were microscoped and turbidity was measured. Nanobacteria control dishes were prepared by adding 1 part Nanobacteria suspension in 20% FBS-90% DMEM and 1 part of DMEM. Baseline measurement and growth measurement 8 days after were done using turbidometer. Negative control consisted of 20% FBS-90% DMEM. Chemical controls were prepared by adding 1 part chemical dilution+1 part 20% FBS-90% DMEM and were incubated for 8 days as described above. At day 8 dishes were observed for formation of precipitates. The inhibition percentage (%) was calculated as: (turbidity of nanos with chemical)/(turbidity of Nanobacteria control)*100%.

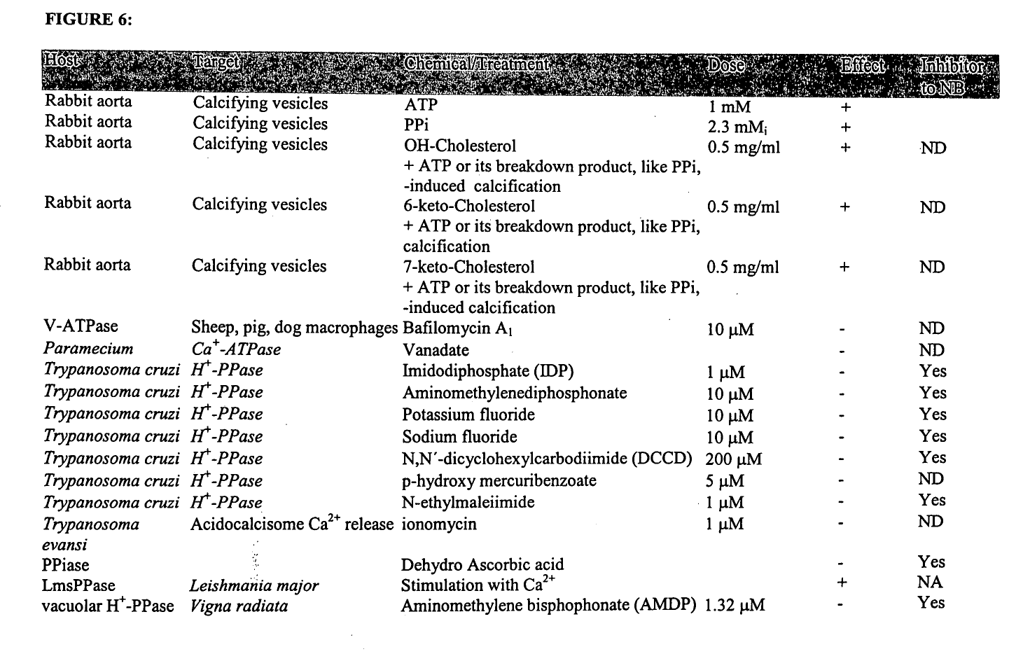

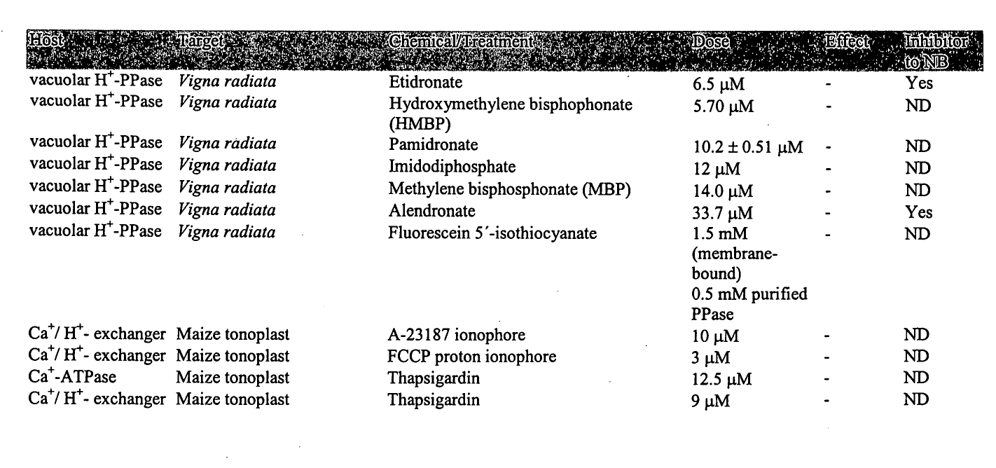

FIG. 6 depicts the effect of agents inhibiting or activating known vacuolar H⁺-PPase and Ca⁺-ATPase. These vacuolar pumps concentrate calcium and phosphate into vacuoles resulting in up to molar concentrations in some systems. Such concentrations may provoke CaP crystallization and thus inhibitors of these pumps can have anti-calcification effect.

The formulations of the present invention comprise compositions made by combining calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents. Such compositions can comprise calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents in a quantitative ratio from about 100:1 to about 0.01:1 by weight, to from about 10:1 to about 0.10:1 by weight. Compositions of the present invention may further contain 1:1 weight ratios of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents.

Total doses of the calcium chelators, according embodiments of the present invention may range from 0.1-3,000 mg/day, to 10-2,000 mg/day, to 100-1,500 mg/day.

Total doses of the bisphosphonates depend heavily upon the type of bisphosphonate used. For example, alendronate, an aminobisphosphonate, is approximately 700-fold more potent than etidronate, both in vitro and in vivo.

Total doses of the antibiotics, according to embodiments of the present invention may range from 0.01-1,000 mg/day, to 0.1-750 mg/day to 1 to 500 mg/day.

The quantity and doses of each component of the antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents as well as the quantity used in the invention may be varied for different patients and/or treatment conditions.

The compositions of the present invention can be taken in amounts sufficient to provide the desired dosages discussed above.

According to the present invention, and as mentioned above, one of the synergistic effects of the active compounds that make up the composition of the present invention is the ability to achieve improved end results than those that can possibly be achieved with the use of any one of the compounds alone. Such improved results can be obtained by administering a composition of the present invention which comprises a combination of multi-targeted anti-calcification therapy regimen comprising a combination of two or more agents or treatments from one or more classes of the following anti-calcification regimen:

Class 1: A treatment effective against active calcification nidi, including nanobacteria targeted-antibiotics or antimicrobial agents, or anti-metabolites or effective cytostatic agents, or vaccination against nanobacteria, or any physical anti-nanobacteria treatment including light and photodynamic therapies and the combinations therefrom.

Class 2: An agent blocking calcium and phosphate accumulation into a vesicle derived from dead host cells, or acidocacisome-like cell organelle, or nanobacteria or a comparable delineated entity (calcium ATPase and pyrophosphatase pump inhibitors including bisphosphonates, vitamin C, vanadate, fluoride, N-ethylmaleimide, N,N-dicyclohexyl carbodiimide, imidodiphosphate, bafilomycin A or calcimycin or some other antibiotics).

Class 3: An agent effective against calcium phosphate-crystal nucleation and crystal growth (including, e.g., pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom.

Class 4: A calcium phosphate-crystal dissolving agent (any calcium chelator, citrate, lactate and/or other organic and inorganic acidic compounds, some bisphosphonates), and their combinations.

Class 5: Supportive agents dissolving non-mineral components of the stone or calcification, or protecting against blood clotting and thrombosis induced by exposed calcium surface, or improving drug penetration and tissue blood flow, or preventing tissue destruction and improving tissue remodeling and tissue healing, or controlling inflammation and immune response, or preventing calcium-mediated mixed bacterial biofilm formation. These may include administration of bile acid derivatives, terpenes, organic solvents, anti-lipemic drugs, statins, anti-platelet agents, anti-blood clotting agents, non-steroidal anti-inflammatory drugs, immunomodulators, amino acids, vitamins, antioxidants, anti cell death agents, matrix metalloproteinase inhibitors, enzyme systems, antibiotics, fluoride, bisphosphonates, calcium chelators, citrate compounds and calcium-sequestering acids.

Formulations:

The pharmaceutical formulations of the present invention can contain as active ingredients from about 0.5 to about 95.0% wt of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents. This dosage is obtained by mixing the composition of the present invention with different excipients such as agglutinants, disintegrators, lubricants, sliders or just fillers. These excipients include lactose, corn starch, saccharose, magnesium stearate, microcrystalline cellulose, sodium croscarmellose gelatin, cellulose acetophtalate, titanium dioxide, special talc for tablets and polyethylene glycol.

The pharmaceutical composition of the present invention may be administered to humans and animals. The daily dosage of this composition to be used for inhibiting and/or preventing pathological calcification, Nanobacteria, and calcification-induced diseases including, but not limited to, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease), is established between 0.1 to 3,000 mg/day for the calcium chelator/bisphosphonate subsituent, 0.01 to 1,000 mg/day for the antibiotic subsituent, and any therapeutically effective dose, depending on the condition being treated, for the antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents, depending on which calcium chelators, bisphosphonates, antibiotics and/or a combination of amino acids and enzyme systems are present.

The therapeutic composition of the present invention may be packaged in any convenient, appropriate packaging.

As will be appreciated by one knowledgeable in the art, the therapeutic composition of the present invention may be combined or used in combination with other treatments.

The compositions of the invention may be in forms suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing), or as a suppository for rectal dosing.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insulation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of the active ingredients comprising the composition of this invention that is combined with one or more excipients to produce a single dosage form will necessarily vary depending upon the host treated and the particular route of administration. For example, a formulation intended for oral administration to humans may contain the active agent compounded with an appropriate and convenient amount of excipients which may vary from about 5 to about 95 percent by weight of the total composition.

As another example, one embodiment of the present invention contemplates using and administering the calcium chelator, bisphosphonate, antibiotic and combination of amino acids and enzyme systems together in a single dose that can be taken once or more times per day in order to inhibit the growth of Nanobacterium.

In order to use the formulation of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents for the therapeutic treatment (including prophylactic treatment) of mammals, including humans, the composition utilized may be formulated in accordance with standard pharmaceutical practice as a pharmaceutical composition as discussed above. According to this aspect of the invention there is provided a pharmaceutical composition of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents in association with a pharmaceutically acceptable diluent or carrier, wherein the calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents are present in an amount for effectively treating or preventing pathological calcification, Nanobacteria, and calcification-induced diseases.

Administration:

The composition of the present invention can be administered to a patient by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release formulation.

1. Parenteral:

For example, a pharmaceutically acceptable formulation of the composition of the present invention may be formulated for parenteral administration, e.g., for intravenous, subcutaneous, or intramuscular injection. For an injectable formulation, a dose of the composition of the present invention may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulation may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations.

2. Controlled/Extended/Sustained/Prolonged Release Administration

Another aspect of this invention provides methods of treating and/or preventing pathological calcification, Nanobacteria and/or calcification-induced diseases by delivering the composition of the present invention to a patient as a controlled release formulation. As used herein, the terms “controlled” “extended” “sustained” or “prolonged” release of the composition of the present invention will collectively be referred to as “controlled release” and includes continuous or discontinuous, linear or non-linear release of the composition of the present invention.

There are many advantages for a controlled release formulation of the composition of the present invention. Among these are to effectively suppress calcification and Nanobacterial growth during a period when the patient would not be readily able or willing to periodically ingest the composition of the present invention, the composition of the present invention is preferably administered following the evening meal and prior to bedtime in a single dose. The single dose of composition of the present invention preferably is administered via ingestion of one or more controlled release unit dosage forms, so that effective levels are maintained throughout the night.

A sample composition for a controlled release tablet may include, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

More specifically, such a controlled release tablet may include: (a) about 5-20 percent by weight hydroxypropyl methylcellulose having a viscosity of about 10,000 CPS or greater, a substitution rate for the methoxyl group of about 7-30% and a substitution rate for the hydroxypropoxyl group of about 7-20%; (b) about 2-8 percent hydroxypropyl methylcellulose having a viscosity of less than about 100, CPS methyl cellulose, or polyvinyl pyrollidone; (c) about 5-15 percent by weight hydrogenated vegetable oil or stearic acid; and (d) about 30-90% active ingredient.

High viscosity water-soluble 2-hydroxypropyl methyl cellulose (HPMC) is particularly preferred for use in the present tablets and in the controlled-release tablet coating, due to its sustaining properties with respect to release of the compositions of the present invention. A particularly preferred high viscosity HMPC has a nominal viscosity, two percent solution, of about 100,000 CPS, methoxyl content of about 19-24, a hydroxypropyl content of about 7-12 percent, and a particle size where at least 90% passes through a USS 100 mesh screen. (Methocel® K100MCR). Low viscosity HPMC is preferred as the binder component of the tablet. A particularly preferred low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

The viscosities reported herein are measured in centipoises (cps or cP), as measured in a 2% by weight aqueous solution of the cellulose either at 20° C. using a rotational viscometer. A “high viscosity” cellulose ether possesses a viscosity of at least about 10,000 cps i.e., about 50,000-100,000 cps. A low-viscosity cellulose ether possesses a viscosity of less than about 100 cps, i.e., about 10-100 cps.

“Water soluble” for purposes of this application means that two grams of powdered cellulose ether can be dispersed by stirring into 100 grams of water at a temperature between 0° C.-100° C. to provide a substantially clear, stable aqueous composition or dispersion (when the dispersion is brought to 20° C.).

Useful hydrophobic components include natural and synthetic waxes such as beeswax, carnauba wax, paraffin, spermaceti, as well as synthetic waxes, hydrogenated vegetable oils, fatty acids, fatty alcohols and the like.

The controlled release tablets may be formulated to contain 0.1 to 3,000 mg of calcium chelator or bisphosphonate, 0.01 to 1,000 mg of antibiotic, and any quantity of each component of the nutraceutical powder determined by the patients and/or treatment conditions, depending on the particular compositions used, and are ingested orally.

Preferably, these tablets will release about 10-35 wt-% of the total active ingredients of the present invention within about 2 hours in an in vitro dissolution test, and about 40-70 wt-% of the total active ingredients of the present invention in eight hours.

These controlled released tablets can also be coated so as to further prolong the release of the active ingredients of the present invention into the gastrointestinal tract, or to prevent its release into the stomach, in order to prevent or attenuate the gastrointestinal side effects which can accompany administration of calcium chelators such as EDTA.

For example, coatings comprising a major portion of a polymeric material having a high degree of swelling on contact with water or other aqueous liquids can be used to further prolong the release of the calcium chelators such as EDTA from the tablets core. Such polymers include, inter alia, cross-linked sodium carboxymethylcellulose (Acdisol-FMC), cross-linked hydroxypropylcellulose, hydroxymethylpropylcellulose, e.g., Methocel® K15M, Dow Chem. Co., carboxymethylamide, potassium methylacrylate divinylbenzene copolymer, polymethyl methacrylate, cross-linked polyvinylpyrrolidine, high molecular weight polyvinylalcohol, and the like. Hydroxypropylmethyl cellulose is available in a variety of molecular weights/viscosity grades from Dow Chemical Co. under the Methocel® designation. See also, Alderman (U.S. Pat. No. 4,704,285). These polymers may be dissolved in suitable volatile solvents, along with dyes, lubricants, flavorings and the like, and coated onto the prolonged release tablets, e.g., in amounts equal to 0.1-5% of the total tablet weight, by methods well known to the art. For example, see Remington's Pharmaceutical Sciences, A. Osol, ed., Mack Publishing Co., Easton, Pa. (16th ed. 1980) at pages 1585-1593.

Release rates can be profiled dependent upon pH conditions for release into the system.

Enteric coatings can also be provided to the prolonged release tablets to prevent release of the active ingredients of the present invention until the tablet reaches the intestinal tract. Such coatings comprise mixtures of fats and fatty acids, shellac and shellac derivatives and the cellulose acid phthlates, e.g., those having a free carboxyl consent of 9-15%. See, Remington's at page 1590, and Zeitova et al. (U.S. Pat. No. 4,432,966), for descriptions of suitable enteric coating compositions.

3. Films

This invention further provides a prophylaxis for or method of treating a patient following an invasive surgical procedure comprising administering biodegradable, biocompatible polymeric film comprising the composition of the present invention to a patient. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers, and between about 60 and about 300 micrometers are useful in the manufacture of therapeutic implants for insertion into a patient's body. The films can be administered to the patient in a manner similar to methods used in adhesion surgeries. For example, a calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents film formulation can be sprayed or dropped onto a site during surgery, or a formed film can be placed over the selected tissue site. In an alternative embodiment, the film can be used as controlled release coating on a medical device such as a stent, or hip replacement, as is discussed in further detail below.

Either biodegradable or nonbiodegradable polymers may be used to fabricate implants in which the calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents are uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

A plasticizer may be incorporated in the biodegradable film to make it softer and more pliable for applications where direct contact with a contoured surface is desired.

The polymeric films of this invention can be formed and used as flat sheets, or can be formed into three-dimensional conformations or “shells” molded to fit the contours of the tissue site into which the film is inserted.

To make the polymeric films of this invention, a suitable polymeric material is selected, depending on the degradation time desired for the film. A lower molecular weight, e.g., around 20,000 daltons, 50:50 or 55:45 PLA:PGA copolymer is used when a shorter degradation time is desired. To ensure a selected degradation time, the molecular weights and compositions may be varied.

Polymeric films of this invention may be made by dissolving the selected polymeric material in a solvent such as acetone, chloroform or methylene chloride, using about 20 mL solvent per gram of polymer. The solution is then degassed, preferably under gentle vacuum to remove dissolved air and poured onto a surface, preferably a flat non-stick surface such as BYTAC (Trademark of Norton Performance Plastics, Akron, Ohio) non-stick coated adhesive-backed aluminum foil, glass or TEFLON™ non-stick polymer. The solution is then dried, preferably air-dried, until it is no longer tacky and the liquid appears to be gone. The known density of the polymer may be used to back-calculate the volume of solution needed to produce a film of the desired thickness.

Films may also be made by heat pressing and melt forming/drawing methods known to the art. For example, thicker films can be pressed to form thinner films, and can be drawn out after heating and pulled over forms of the desired shapes, or pulled against a mold by vacuum pressure.

The amount of the composition of the present invention to be incorporated into the polymeric films of this invention is an amount effective to show a measurable effect in treating calcification, Nanobacteria and/or calcification-induced diseases. The composition of the present invention can be incorporated into the film by various techniques such as by solution methods, suspension methods, or melt pressing.

Solid implants comprising the composition of the present invention can also be made into various shapes other than films by injection molding or extrusion techniques. For example, the implant can comprise a core material such as ethylene/vinyl acetate copolymer, and a vinyl acetate content of 20% by weight or more and which functions as a matrix for the composition of the present invention, in a quantity which is sufficient for a controlled release of the composition of the present invention, and a membrane which encases the core material and also consists of EVA material and an acetate content of less than 20% by weight. The implant can be obtained, for example, by means of a co-axial extrusion process, a method in which the two EVA polymers are extruded co-axially with the aid of a co-axial extrusion head. The co-axial extrusion process is art known per se so that it will not be gone into further within the scope of this description.

4. Transdermal Patch Device

Transdermal delivery, involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Characteristically, these devices contain a drug impermeable backing layer which defines the outer surface of the device and a permeable skin attaching membrane, such as an adhesive layer, sealed to the barrier layer in such a way as to create a reservoir between them in which the therapeutic agent is placed. In one embodiment of the present invention a formulation of the composition of the present invention is introduced into the reservoir of a transdermal patch.

5. Medical Devices

Another embodiment contemplates the incorporation of the composition of the present invention into a medical device that is then positioned to a desired target location within the body, whereupon the composition of the present invention elutes from the medical device. As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen. Medical devices may include stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), mesh repair materials, a Dracon vascular particle orthopedic metallic plates, rods and screws and vascular sutures. Thus, by way of example, the present invention will be described in relation to vascular stents. However, it should be understood that the following embodiments relate to any medical device incorporating the composition of the present invention, and is not limited to any particular type of medical device.

The devices of this invention provide a therapeutically effective amount of the composition of the present invention to a targeted site such as a diseased or injured bodily tissue or organ. The precise desired therapeutic effect will vary according to the condition to be treated, the formulation to be administered, and a variety of other factors that are appreciated by those of ordinary skill in the art. The amount of the composition of the present invention needed to practice the claimed invention also varies with the nature of the device used. For purposes of this invention, “elution” refers to any process of release that involves extraction or release by direct contact of the coating with bodily fluids.

In one embodiment, and by way of example, the medical device to be coated with the composition of the present invention is a stent or catheter for performing or facilitating a medical procedure. Accordingly, the present invention may be used in conjunction with any suitable or desired set of stent components and accessories, and it encompasses any of a multitude of stent designs. These stent designs may include for example a basic solid or tubular flexible stent member or a balloon catheter stent, up to complex devices including multiple tubes or multiple extruded lumens, as well as various accessories such as guide wires, probes, ultrasound, optic fiber, electrophysiology, blood pressure or chemical sampling components. In other words, the present invention may be used in conjunction with any suitable stent or catheter design, and is not limited to a particular type of catheter.

In another embodiment, the stent can be designed to have pores for the delivery of the composition of the present invention to the desired bodily location. Briefly, this may involve providing a powdered metal or polymeric material, subjecting the powder to high pressure to form a compact, sintering the compact to form a final porous metal or polymer, forming a stent from the porous metal and, optionally, loading at least the composition of the present invention (and optionally one or more additional drugs) into the pores. For example, the stent may be impregnated with the composition of the present invention and optionally one or more additional drugs by any known process in the art, including high pressure loading in which the stent is placed in a bath of the desired drug or drugs and subjected to high pressure or, alternatively, subjected to a vacuum. The drug(s) may be carried in a volatile or non-volatile solution. In the case of a volatile solution, following loading of the drug(s), the volatile carrier solution may be volatilized. In the case of the vacuum, the air in the pores of the metal stent is evacuated and replaced by the drug-containing solution. Alternatively, rather than loading the porous stent with the drug, the stent is instead implanted in the desired bodily location, and then the drug is injected through a delivery tubing to the hollow stent and then out the pores in the stent to the desired location.

In another embodiment, the stent can be designed to contain reservoirs or channels which could be loaded with the composition of the present. A coating or membrane of biocompatible material could be applied over the reservoirs which would control the diffusion of the drug from the reservoirs to the artery wall. One advantage of this system is that the properties of the coating can be optimized for achieving superior biocompatibility and adhesion properties, without the additional requirement of being ale to load and release the drug. The size, shape, position, and number of reservoirs can be used to control the amount of drug, and therefore the dose delivered.

The stent can be made of virtually any biocompatible material having physical properties suitable for the design, and can be biodegradable or nonbiodegradable. The material can be either elastic or inelastic, depending upon the flexibility or elasticity of the polymer layers to be applied over it. Accordingly, the medical devices of this invention can be prepared in general from a variety of materials including ordinary metals, shape memory alloys, various plastics and polymers, carbons or carbon fibers, cellulose acetate, cellulose nitrate, silicone and the like.

For example, a medical device, such as but not limited to a stent, according to this invention can be composed of polymeric or metallic structural elements onto which a matrix is applied or the stent can be a composite of the matrix intermixed with a polymer.

Suitable biocompatible metals for fabricating the expandable stent include high grade stainless steel, titanium alloys including NiTi (a nickel-titanium based alloy referred to as Nitinol), cobalt alloys including cobalt-chromium-nickel alloys such as Elgiloy® and Phynox®, a Niobium-Titanium (NbTi) based alloy, tantalum, gold, and platinum-iridium.

Suitable nonmetallic biocompatible materials include, but are not limited to, polyamides, polyolefins (e.g., polypropylene, polyethylene etc.), nonabsorbable polyesters (i.e. polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone, etc. and blends thereof).

In one embodiment, the medical device, such as a stent, is coated with a matrix. The matrix used to coat the stent, according to this invention may be prepared from a variety of materials. A primary requirement for the matrix is that it be sufficiently elastic and flexible to remain unruptured on the exposed surfaces of the stent.

(A) Naturally Occurring Materials

The matrix may be selected from naturally occurring substances such as film-forming polymeric biomolecules that may be enzymatically degraded in the human body or are hydrolytically unstable in the human body such as fibrin, fibrinogen, heparin, collagen, elastin, and absorbable biocompatable polysaccharides such as chitosan, starch, fatty acids (and esters thereof), glucoso-glycans, hyaluronic acid, carbon, laminin, and cellulose.

(B) Synthetic Materials

In one embodiment, matrix that is used to coat the stent may be selected from any biocompatible polymeric material capable of holding the composition of the present invention. The polymer chosen must be a polymer that is biocompatible and minimizes irritation to the vessel wall when the stent is implanted. The polymer may be either a biostable or a bioabsorbable polymer depending on the desired rate of release or the desired degree of polymer stability.

Suitable materials for preparing a polymer matrix include, but are not limited to, polycarboxylic acids, cellulosic polymers, silicone adhesive, fibrin, gelatin, polyvinylpyrrolidone, maleic anhydride polymers, polyamides, polyvinyl alcohols, polyethylene glycols, polyethylene oxides, glycosaminoglycans, polysaccharides, polyesters, poly(amino acids)polyurethanes, segmented polyurethane-urea/heparin, silicons, polyorthoesters, polyanhydrides, polycarbonates, polypropylenes, poly-L-lactic acids, polyglycolic acids, polycaprolactones, polyhydroxybutyrate valerates, polyacrylamides, polyethers, polyalkylenes oxalates, polyamides, poly(iminocarbonates), polyoxaesters, polyamidoesters, polyoxaesters containing amido groups, polyphosphazenes, vinyl halide polymers, polyvinylidene halides, polyacrylonitrile, polyvinyl ketones, polyvinyl aromatics (e.g., polystyrene), etheylene-methyl methacrylate copolymers, acrylonitrile-styrene copolymers, ABS resins and ethylene-vinyl acetate copolymers; polyamides, such as Nylon 66 and polycaprolactam; alkyl resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e. carboxymethyl cellulose and hydoxyalkyl celluloses) and mixtures and copolymers thereof.

The polymers used for coatings are preferably film-forming polymers that have molecular weight high enough as to not be waxy or tacky. The polymers also preferably adhere to the stent and are not so readily deformable after deposition on the stent as to be able to be displaced by hemodynamic stresses. The polymers molecular weight are preferably high enough to provide sufficient toughness so that the polymers will not be rubbed off during handling or deployment of the stent and will not crack during expansion of the stent.

In one embodiment, the matrix coating can include a blend of a first co-polymer having a first, high release rate and a second co-polymer having a second, lower release rate relative to the first release rate. The first and second copolymers are preferably erodible or biodegradable. In one embodiment, the first copolymer is more hydrophilic than the second copolymer. For example, the first copolymer can include a polylactic acid/polyethylene oxide (PLA-PEO) copolymer and the second copolymer can include a polylactic acid/polycaprolactone (PLA-PCL) copolymer. Formation of PLA-PEO and PLA-PCL copolymers is well known to those skilled in the art. The relative amounts and dosage rates of the composition of the present invention delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer.

Alternatively, a top coating can be applied to delay release of the active ingredients, or could be used as the matrix for the delivery of a different pharmaceutically active material. For example, layering of coatings of fast and slow hydrolyzing copolymers can be used to stage release of the drug or to control release of different agents placed in different layers. Polymers with different solubilities in solvents can be used to build up different polymer layers that may be used to deliver different active ingredients or control the release profile of a drug. For example since ε-caprolactone-co-lactide elastomers are soluble in ethyl acetate and ε-caprolactone-co-glycolide elastomers are not soluble in ethyl acetate. A first layer of ε-caprolactone-co-glycolide elastomer containing a drug can be over coated with ε-caprolactone-co-glycolide elastomer using a coating solution made with ethyl acetate as the solvent. As will be readily appreciated by those skilled in the art numerous layering approaches can be used to provide the desired delivery of the composition of the present invention.

In one embodiment the coating is formulated by mixing the composition of the present invention and optionally one or more additional therapeutic agents with the coating polymers in a coating mixture. The composition of the present invention and the therapeutic agent may be present as a liquid, a finely divided solid, or any other appropriate physical form. Optionally, the mixture may include one or more additives, e.g., nontoxic auxiliary substances such as diluents, carriers, excipients, stabilizers or the like. Other suitable additives may be formulated with the polymer and the composition of the present invention and pharmaceutically active agent or compound. For example hydrophilic polymers selected from the previously described lists of biocompatible film forming polymers may be added to a biocompatible hydrophobic coating to modify the release profile (or a hydrophobic polymer may be added to a hydrophilic coating to modify the release profile). One example would be adding a hydrophilic polymer selected from the group consisting of polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the composition of the present invention and the therapeutic agents.

(C) Biodegradable Matrix

In one embodiment, the matrix is a synthetic or naturally occurring biodegradable polymer such as aliphatic and hydroxy polymers of lactic acid, glycolic acid, mixed polymers and blends, polyhydroxybutyrates and polyhydroxy-valeriates and corresponding blends, or polydioxanon, modified starch, gelatine, modified cellulose, caprolactaine polymers, polyacrylic acid, polymethacrylic acid or derivatives thereof, which will not alter the structure or function of the medical device. Such biodegradable polymers will disintegrate in a controlled manner (depending on the characteristics of the carrier material and the thickness of the layer(s) thereof), with consequent slow release of the composition of the present invention incorporated therein, while in contact with blood or other body fluids.

(D) Application of the Matrix to the Medical Device

In accordance with one embodiment of the present invention, the composition of the present invention is applied as an integral part of a coating on at least the exterior surface of the stent. The solution is applied to the stent and the solvent is allowed to evaporate, thereby leaving on the stent surface a coating of the polymer and the therapeutic substance. Typically, the solution can be applied to the stent by any suitable means such as, for example, by immersion, spraying, or deposition by plasma or vapor deposition. In order to coat a medical device such as a stent, the stent is dipped or sprayed with a liquid solution of the matrix of moderate viscosity. After each layer is applied, the stent is dried before application of the next layer. In one embodiment, a thin, paint-like matrix coating does not exceed an overall thickness of 100 microns. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the stent. In either a coating applied by spraying or by immersion, multiple application steps are generally desirable to provide improved coating uniformity and improved control over the amount of therapeutic substance to be applied to the stent. The amount of the composition of the present invention to be included on the stent can be readily controlled by applying multiple thin coats of the solution while allowing it to dry between coats. The overall coating should be thin enough so that it will not significantly increase the profile of the stent for intravascular delivery by catheter. The adhesion of the coating and the rate at which the composition of the present invention is delivered can be controlled by the selection of an appropriate bioabsorbable or biostable polymer and by the ratio of composition of the present invention to polymer in the solution.

In order to provide the coated stent according to this embodiment, a solution which includes a solvent, a polymer dissolved in the solvent, the composition of the present invention dispersed in the solvent, and optionally a cross-linking agent, is first prepared. It is important to choose a solvent and polymer that are mutually compatible with the composition of the present invention. It is essential that the solvent is capable of placing the polymer into solution at the concentration desired in the solution. It is also essential that the solvent and polymer chosen do not chemically alter the therapeutic character of the composition of the present invention. However, the composition of the present invention only needs to be dispersed throughout the solvent so that it may be either in a true solution with the solvent or dispersed in fine particles in the solvent. Preferable conditions for the coating application are when the polymer and composition of the present invention have a common solvent. This provides a wet coating that is a true solution. Less desirable, yet still usable are coatings that contain the composition of the present invention as a solid dispersion in a solution of the polymer in solvent. Under the dispersion conditions, care must be taken if a slotted or perforated stent is used to ensure that the particle size of the dispersed pharmaceutical powder, both the primary powder size and its aggregates and agglomerates, is small enough not to cause an irregular coating surface or to clog the slots or perforations of the stent. In cases where a dispersion is applied to the stent and it is desired to improve the smoothness of the coating surface or ensure that all particles of the drug are fully encapsulated in the polymer, or in cases where it is desirable to slow the release rate of the drug, deposited either from dispersion or solution, a clear (polymer only) top coat of the same polymer used to provide controlled release of the drug or another polymer can be applied that further restricts the diffusion of the drug out of the coating.

The composition coats the exterior and interior surfaces of the stent and, as it solidifies, encapsulates these surfaces in the polymer/composition of the present invention formulation. The dried stent thus includes a coating of the composition of the present invention on its surfaces. Preferably, the immersion methods are adapted such that the solution or suspension does not completely fill the interior of the stent or block the orifice. Methods are known in the art to prevent such an occurrence, including adapting the surface tension of the solvent used to prepare the composition, clearing the lumen after immersion, and placement of an inner member with a diameter smaller than the lumen in such a way that a passageway exists between all surfaces of the stent and the inner member. An alternative to dipping the distal end of the stent is to spray-coat the exterior and interior surfaces with a vaporized form of the composition comprising the composition of the present invention.

In one embodiment, the matrix is chosen such that it adheres tightly to the surface of the stent or synthetic graft. This can be accomplished, for example, by applying the matrix in successive thin layers. Each layer of matrix may incorporate the antibodies. Alternatively, composition of the present invention may be applied only to the layer in direct contact with the vessel lumen. Different types of matrices may be applied successively in succeeding layers.

The solvent is chosen such that there is the proper balance of viscosity, deposition level of the polymer, solubility of the pharmaceutical agent, wetting of the stent and evaporation rate of the solvent to properly coat the stents. In the preferred embodiment, the solvent is chosen such the composition of the present invention and the polymer are both soluble in the solvent. In some cases, the solvent must be chosen such that the coating polymer is soluble in the solvent and such that the pharmaceutical agent is dispersed in the polymer solution in the solvent. In that case the solvent chosen must be able to suspend small particles of the composition of the present invention without causing them to aggregate or agglomerate into collections of particles that would clog the slots of the stent when applied. Although the goal is to dry the solvent completely from the coating during processing, it is a great advantage for the solvent to be non-toxic, non-carcinogenic and environmentally benign. Mixed solvent systems can also be used to control viscosity and evaporation rates. In all cases, the solvent must not react with or inactivate the composition of the present invention or react with the coating polymer. Preferred solvents include, but are not limited to, acetone, N-methylpyrrolidone (NMP), dimethyl sulfoxide (DMSO), toluene, xylene, methylene chloride, chloroform, 1,1,2-trichloroethane (TCE), various freons, dioxane, ethyl acetate, tetrahydrofuran (THF), dimethylformamide (DMF), dimethylacetamide (DMAC), water, and buffered saline.

In one embodiment, a stent is coated with a mixture of a pre-polymer, cross-linking agents and the composition of the present invention, and then subjected to a curing step in which the pre-polymer and cross-linking agents cooperate to produce a cured polymer matrix containing the composition of the present invention. The curing process involves evaporation of the solvent and the curing and cross-linking of the polymer. Certain silicone materials can be cured at relatively low temperatures, (i.e., room temperature to 50° C.) in what is known as a room temperature vulcanization (RTV) process. Of course, the time and temperature may vary with particular silicones, cross-linkers and biologically active species.

Generally, the amount of coating to be placed on the catheter will vary with the polymer, and may range from about 0.1 to 40 percent of the total weight of the catheter after coating. The polymer coatings may be applied in one or more coating steps depending on the amount of polymer to be applied.

(E) Addition of the Composition of the Present Invention to the Matrix

The composition of the present invention can be incorporated into the matrix, either covalently or noncovalently, wherein the coating layer provides for the controlled release of the composition of the present invention from the coating layer. The composition of the present invention may be incorporated into each layer of matrix by mixing the composition of the present invention with the matrix coating solution. Alternatively, the composition of the present invention may be covalently or noncovalently coated onto the last layer of matrix that is applied to the medical device. The desired release rate profile of the composition of the present invention from the device can be tailored by varying the coating thickness, the radial distribution (layer to layer) of the composition of the present invention, the mixing method, the amount of the composition of the present invention, the combination of different matrix polymer materials at different layers, and the crosslink density of the polymeric material, as discussed below.

In one embodiment, the composition of the present invention is added to a solution containing the matrix. For example, the composition of the present invention can be incubated with a solution containing a polymer at an appropriate concentration of the composition of the present invention. It will be appreciated that the concentration of the composition of the present invention will vary and that one of ordinary skill in the art could determine the optimal concentration without undue experimentation. The composition of the present invention/polymer mixture is then applied to the device by any of the methods described herein.

The ratio of the composition of the present invention to polymer in the solution will depend on the efficacy of the polymer in securing the composition of the present invention onto the stent and the rate at which the coating is to release the composition of the present invention to the tissue of the blood vessel. More polymer may be needed if it has relatively poor efficacy in retaining the composition of the present invention on the stent and more polymer may be needed in order to provide an elution matrix that limits the elution of a very soluble composition of the present invention. A wide ratio of composition of the present invention to polymer could therefore be appropriate and could range from about 10:1 to about 1:100.

(F) Deposition of the Composition of the Present Invention onto a Medical Device

In another embodiment, a medical device of this invention such as a stent comprises at least one layer of the composition of the present invention deposited on at least a portion of a coating layer of the stent. If desired, a porous layer can be deposited over the composition of the present invention layer, wherein the porous layer includes a polymer and provides for the controlled release of the composition of the present invention therethrough and further avoids degradation of the composition of the present invention. Methods of coating a stent according to this embodiment is disclosed in U.S. Pat. No. 6,299,604, which is specifically incorporated herein by reference.

In yet another embodiment, the composition of the present invention is covalently coupled to the matrix. In one embodiment, the composition of the present invention can be covalently coupled to the matrix through the use of hetero- or homobifunctional linker molecules. The use of linker molecules in connection with the present invention typically involves covalently coupling the linker molecules to the matrix after it is adhered to the stent. After covalent coupling to the matrix, the linker molecules provide the matrix with a number of functionally active groups that can be used to covalently couple one or more types of composition of the present invention. The linker molecules may be coupled to the matrix directly (i.e., through the carboxyl groups), or through well-known coupling chemistries, such as, esterification, amidation, and acylation. For example, the linker molecule could be a polyamine functional polymer such as polyethyleneimine (PEI), polyallylamine (PALLA) or polyethyleneglycol (PEG). A variety of PEG derivatives, e.g., mPEG-succinimidyl propionate or mPEG-N-hydroxysuccinimide, together with protocols for covalent coupling, are commercially available from Shearwater Corporation, Birmingham, Ala. (See also, Weiner, et al., J. Biochem. Biophys. Methods, 45:211-219 (2000), incorporated herein by reference). It will be appreciated that the selection of the particular coupling agent may depend on the type of delivery vehicle used in the composition of the present invention and that such selection may be made without undue experimentation.

(G) Coating a Medical Device with the Composition of the Present Invention

In yet another embodiment, a thin layer of the composition of the present invention is covalently or noncovalently bonded to the exterior surfaces of the stent. In this embodiment, the stent surface is prepared to molecularly receive the composition of the present invention according to methods known in the art. If desired, a porous layer can be deposited over the composition of the present invention layer, wherein the porous layer includes a polymer and provides for the controlled release of the composition of the present invention therethrough and further avoids degradation of the composition of the present invention.

(H) Compounded Medical Devices

In an alternative embodiment of a medical device according to the invention, the composition of the present invention is provided throughout the body of the medical device by mixing and compounding the composition of the present invention directly into the medical device polymer melt before forming the medical device. For example, the composition of the present invention can be compounded into materials such as silicone, rubber or urethane. The compounded material is then processed by conventional method such as extrusion, transfer molding or casting to form a particular configuration. The medical device resulting from this process benefits by having the composition of the present invention dispersed throughout the entire medical device body. Thus, the composition of the present invention is present at the outer surface of the medical device when the medical device is in contact with bodily tissues, organs or fluids and acts to modulate an immune response.

Treatment Protocol:

Another embodiment of the present invention contemplates the use and administration of the calcium chelator, bisphosphonate, antibiotic and combination of amino acids and enzyme systems where each compound is administered separately and sequentially once or more times per day.

According to this aspect of the invention there is provided a protocol for the separate and sequential administration of pharmaceutical compositions of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents, wherein the calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents are present in an amount for effectively treating or preventing pathological calcification, Nanobacteria, and calcification-induced diseases including, but not limited to, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease), in an individual in need thereof.

The protocol of the present invention can be administered to a patient by any available and effective delivery systems including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release formulation.

For example, the pharmaceutical compositions of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents, in dosages described in detail above, may be sequentially administered until a therapeutically effective quantity is administered for the treatment or prevention of pathological calcification, Nanobacteria, and calcification-induced diseases.

In one embodiment of the protocol, the patient begins the day with a normal breakfast. The patient is instructed to avoid taking any mineral supplements unless directed to do so by their physician because minerals may decrease the effectiveness of the treatment. The patient is then instructed to eat a good lunch and a very light supper, preferably no later than 6:30 p.m., as a heavy dinner will increase the serum concentration of proteins, fats and mineral ions, which may interfere with the treatment. At bedtime, the combination of amino acids and enzyme systems is orally administered and followed by the antibiotic, which is orally administered. Then, the calcium chelator is administered via suppository.

The initial treatment for calcification, Nanobacteria and calcification-induced diseases according to this embodiment of the protocol is to be carried out on a daily basis for a period of four to six months. Thereafter, the patient is instructed to undergo maintenance therapy by carrying out the described protocol for three days each month.

While undergoing treatment, patients should be monitored at least on a monthly basis. Patients should also receive adequate water hydration daily. Patients with other comorbidities or decreased renal function should be monitored appropriately. Diabetic insulin and hypoglycemic medications may need to be decreased.

The initial treatment for calcification, Nanobacteria and calcification-induced diseases according to this embodiment of the protocol is to be carried out on a daily basis for a period of four to six months. Thereafter, the patient is instructed to undergo maintenance therapy by carrying out the described protocol for three days each month.

While undergoing treatment, patients should be monitored at least on a monthly basis. Patients should also receive adequate water hydration daily. Patients with other comorbidities or decreased renal function should be monitored appropriately. Diabetic insulin and hypoglycemic medications may need to be decreased.

In another embodiment of the treatment protocol, a patient is instructed, prior to going to bed, to mix approximately 5 cm³ of the supportive agents, also referred to herein as the neutroceutical powder, in water, juice (e.g., apple or orange juice) or other suitable liquid prior to being administered. Thereafter, the patient is instructed to orally consume the nutraceutical powder solution. In this embodiment, the patient is also instructed to orally consume approximately 500 mg of tetracycline HCl that had been formulated as a capsule before administration. Next, the patient is instructed to rectally insert approximately 1500 mg of ethylenediaminetetraacetic acid disodium salt (EDTA-sequestrant) that had been formulated as a suppository before administration. Once the three components of the composition were administered, the patient was instructed to lie down flat and fall asleep.

Variations in the above treatment protocol can readily be made. In other embodiments, for example, the order in which the components are administered can be altered. Similarly, in differing embodiments, different quantities of each component may be employed and/or the components may individually or collectively formulated in different manners as warranted by prevailing conditions or patient needs.

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A method for treating or preventing pathological calcification comprising the steps of administering therapeutically effective amounts of at least one of calcium chelators, bisphosphonates, antibiotics, antimicrobial agents, cytostatic agents, calcium ATPase and pyrophosphatase pump inhibitors, calcium phosphate-crystal dissolving agents, and agents effective against calcium phosphate-crystal nucleation and crystal growth.

2. A method for reducing Nanobacteria, comprising the steps of administering therapeutically effective amounts of at least one of calcium chelators, bisphosphonates, antibiotics, antimicrobial agents, cytostatic agents, calcium ATPase and pyrophosphatase pump inhibitors, calcium phosphate-crystal dissolving agents, and agents effective against calcium phosphate-crystal nucleation and crystal growth.

3. A method for treating at least one of calcification-induced diseases, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) in a patient, comprising the steps of delivering to said patient a composition comprising at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents in an amount effective to reduce and/or prevent calcification-induced diseases, Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease).

4. A method of reducing Nanobacteria, comprising the steps of administering a pharmaceutical composition in an amount that destroys a Nanobacteria calcific biofilm and inhibits enzymatic reactions that allow Nanobacteria to reproduce.

5. The method of claim 4, where the Nanobacteria calcific biofilm is destroyed by calcium chelators.

6. The method of claim 4, where the enzymatic reactions are inhibited by antibiotics.

7. The methods of claims 1 and 2, further comprising the steps of administering a combination of supportive agents.

8. The methods of claims 1, 2, and 5 wherein said calcium chelator is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

9. The methods of claims 1 and 2 wherein said bisphosphonate is selected from at least one of alendronate, clodronate, ibandronate, incadronate, neridronate, palmidronate, risedronate, tiludronate, zoledronate, etidronate, oxidronate, and pharmaceutically acceptable salts thereof.

10. The methods of claims 1, 2, and 6 wherein said antibiotic is selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof.

11. The methods of claims 1 and 2, wherein said calcium ATPase and pyrophosphatase pump inhibitor is selected from at least one of bisphosphonates, vitamin C, vanadate, fluoride, N-ethylmaleimide, N,N-dicyclohexylcarbodiimide, imidodiphosphate, bafilomycin A, calcimycin, or other antibiotics.

12. The methods of claims 1 and 2, wherein said calcium phosphate-crystal dissolving agent is selected from at least one of calcium chelators, citrate, lactate, bisphophonates, or other organic and inorganic acidic compounds, including sodium and potassium salts, magnesium citrate, phosphocitrate and other complexes of citrate.

13. The methods of claims 1 and 2, wherein said agent effective against calcium phosphate-crystal nucleation and crystal growth is selected from at least one of pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom.

14. The method of claim 7, wherein said supportive agent is selected from at least one of bile acid derivatives, terpenes, organic solvents, anti-lipemic drugs, statins, anti-platelet agents, anti-blood clotting agents, non-steroidal anti-inflammatory drugs, immunomodulators, amino acids, vitamins, antioxidants, anti cell death agents, matrix metalloproteinase inhibitors, enzyme systems, antibiotics, fluoride, bisphosphonates, calcium chelators, citrate compounds and calcium-sequestering acids.

15. The methods of claims 1, 2, and 5, wherein said calcium chelator is administered orally.

16. The methods of claims 1, 2, and 5 wherein said calcium chelator is delivered to said patient as a controlled/sustained/extended/prolonged release composition.

17. The method of claim 16, wherein said controlled/sustained/extended/prolonged release composition comprises a flowable thermoplastic polymer composition comprising a biocompatible polymer, a biocompatible solvent, and at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents and said controlled/sustained/extended/prolonged release composition is delivered to a bodily tissue or fluid in said patient, wherein the amounts of the polymer and the solvent are effective to form a biodegradable polymer matrix containing at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and combination of supportive agents in situ when said composition contacts said bodily fluid tissue or fluid.

18. The method of claim 17, wherein said polymer is one of a poly(alkylene glycol) or a polysaccharide.

19. The method of claim 17, wherein said biocompatible polymer is selected from at least one of polylactides, polyglycolides, polyanhydrides, polyorthoesters, polycaprolactones, polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, and copolymers and combinations thereof.

20. The method of claim 17, wherein said biodegradable polymer matrix releases at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents by diffusion, erosion, or a combination of diffusion or erosion as the polymer matrix biodegrades in said patient.

21. The method of claim 17, wherein said calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents are added to said polymer composition prior to administration such that said solid polymer matrix further contains at least one of said calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents.

22. The method of claim 16, wherein said controlled/sustained/extended/prolonged release composition is in film form.

23. The method of claim 16, wherein said controlled/sustained/extended/prolonged release is in tablet form.

24. A composition for the treatment, reduction or prevention of pathological calcification, Nanobacteria or calcification-induced diseases, comprising at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents.

25. The composition of claim 24, wherein said calcium chelators are selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

26. The composition of claim 24, wherein said bisphosphonates are selected from at least one of alendronate, clodronate, ibandronate, incadronate, neridronate, palmidronate, risedronate, tiludronate, zoledronate, etidronate, oxidronate, and pharmaceutically acceptable salts thereof.

27. The composition of claim 24, wherein said antibiotics are selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof.

28. The composition of claim 27, wherein said beta-lactam antibiotics are selected from at least one of penicillin, phenethicillin, ampicillin, aziocillin, bacmpicillin, carbenicillin, cylclacillin, mezlocillin, piperacillin, epicillin, hetacillin, cloxacillin, dicloxacillin, methicillin, nafcillin, oxacillin, and pharmaceutically acceptable salts thereof.

29. The composition of claim 27, wherein said aminoglycoside antibiotics are selected from at least one of streptomycin, kanamycin, gentamycin, amikacin, neomycin, pardomycin, tobramycin, viomycin, and pharmaceutically acceptable salts thereof.

30. The composition of claim 27, where said tetracyclines are selected from at least one of tetracycline, chlortetracycline, demeclocycline, doxycycline, methacycline, oxytetracycline, rolitetracycline, minocycline, sancycline and pharmaceutically acceptable salts thereof.

31. The composition of claim 24, wherein said calcium ATPase and pyrophosphatase pump inhibitor is selected from at least one of bisphosphonates, vitamin C, vanadate, fluoride, N-ethylmaleimide, N,N-dicyclohexylcarbodiimide, imidodiphosphate, bafilomycin A, calcimycin, or other antibiotics.

32. The composition of claim 24, wherein said calcium phosphate-crystal dissolving agent is selected from at least one of calcium chelators, citrate, lactate, bisphophonates, or other organic and inorganic acidic compounds, including sodium and potassium salts, magnesium citrate, phosphocitrate and other complexes of citrate.

33. The composition of claim 24, wherein said agent effective against calcium phosphate-crystal nucleation and crystal growth is selected from at least one of pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom.

34. The composition of claim 24, wherein said supportive agent is selected from at least one of bile acid derivatives, terpenes, organic solvents, anti-lipemic drugs, statins, anti-platelet agents, anti-blood clotting agents, non-steroidal anti-inflammatory drugs, immunomodulators, amino acids, vitamins, antioxidants, anti cell death agents, matrix metalloproteinase inhibitors, enzyme systems, antibiotics, fluoride, bisphosphonates, calcium chelators, citrate compounds and calcium-sequestering acids.

35. The composition of claim 24, further comprising a pharmaceutically acceptable carrier, excipient or dilutant.

36. The composition of claim 35, in the form of a capsule, tablet, liquid or powder.

37. The composition of claim 24, wherein said calcium chelators are selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof, said bisphosphonates are selected from at least one of alendronate, clodronate, ibandronate, incadronate, neridronate, palmidronate, risedronate, tiludronate, zoledronate, etidronate, oxidronate, and pharmaceutically acceptable salts thereof, said antibiotics are selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof, said calcium ATPase and pyrophosphatase pump inhibitor is selected from at least one of bisphosphonates, vitamin C, vanadate, fluoride, N-ethylmaleimide, N,N-dicyclohexylcarbodiimide, imidodiphosphate, bafilomycin A, calcimycin, or other antibiotics, said calcium phosphate-crystal dissolving agent is selected from at least one of calcium chelators, citrate, lactate, bisphophonates, or other organic and inorganic acidic compounds, including sodium and potassium salts, magnesium citrate, phosphocitrate and other complexes of citrate, said agent effective against calcium phosphate-crystal nucleation and crystal growth is selected from at least one of pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom, and said supportive agent is selected from at least one of bile acid derivatives, terpenes, organic solvents, anti-lipemic drugs, statins, anti-platelet agents, anti-blood clotting agents, non-steroidal anti-inflammatory drugs, immunomodulators, amino acids, vitamins, antioxidants, anti cell death agents, matrix metalloproteinase inhibitors, enzyme systems, antibiotics, fluoride, bisphosphonates, calcium chelators, citrate compounds and calcium-sequestering acids.

38. The composition of claim 37, wherein said beta-lactam antibiotics are selected from at least one of penicillin, phenethicillin, ampicillin, aziocillin, bacmpicillin, carbenicillin, cylclacillin, mezlocillin, piperacillin, epicillin, hetacillin, cloxacillin, dicloxacillin, methicillin, nafcillin, oxacillin, and pharmaceutically acceptable salts thereof.

39. The composition of claim 37, wherein said aminoglycoside antibiotics are selected from at least one of streptomycin, kanamycin, gentamycin, amikacin, neomycin, pardomycin, tobramycin, viomycin, and pharmaceutically acceptable salts thereof.

40. The composition of claim 37, where said tetracyclines are selected from at least one of tetracycline, chlortetracycline, demeclocycline, doxycycline, methacycline, oxytetracycline, rolitetracycline, minocycline, sancycline and pharmaceutically acceptable salts thereof.

41. The method of claim 22, wherein said film comprises polylactic acid, polyglycolic acid and mixtures and copolymers thereof.

42. An article of manufacture comprising:
(a) a stent body comprising a surface; and
(b) a coating comprising at least one layer disposed over at least a portion of the stent body, wherein the said layer comprises polymer film having at least one biologically active agent dispersed therein.

43. The article of manufacture of claim 42, wherein said biologically active agent is a composition comprising at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents.

44. The composition of claim 24, wherein said calcium chelators are administered in a daily dose within a range from 0.1 to 3,000 mg/day.

45. The composition of claim 24, wherein said calcium chelators are selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof, administered in a daily dose in the range of 10 to 2,000 mg/day.

46. The composition of claim 24, wherein said calcium chelators are selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof, administered in a daily dose in the range of 100 to 1,500 mg/day.

47. The composition of claim 24, wherein said antibiotics are administered in a daily dose in the range of 0.01 to 1,000 mg/day.

48. The composition of claim 24, wherein said antibiotics are selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof, administered in a daily dose in the range of 0.1 to 750 mg/day.

49. The composition of claim 24, wherein said antibiotics are selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof, administered in a daily dose in the range of 1 to 500 mg/day.

50. The composition of claims 48 and 49, wherein said beta-lactam antibiotics are selected from at least one of penicillin, phenethicillin, ampicillin, aziocillin, bacmpicillin, carbenicillin, cylclacillin, mezlocillin, piperacillin, epicillin, hetacillin, cloxacillin, dicloxacillin, methicillin, nafcillin, oxacillin, and pharmaceutically acceptable salts thereof.

51. The composition of claims 48 and 49, wherein said aminoglycoside antibiotics are selected from at least one of streptomycin, kanamycin, gentamycin, amikacin, neomycin, pardomycin, tobramycin, viomycin, and pharmaceutically acceptable salts thereof.

52. The composition of claims 48 and 49, where said tetracyclines are selected from at least one of tetracycline, chlortetracycline, demeclocycline, doxycycline, methacycline, oxytetracycline, rolitetracycline, minocycline, sancycline and pharmaceutically acceptable salts thereof.

53. A controlled/sustained/extended/prolonged release preparation comprising a pharmaceutically active mixture of at least one of calcium chelators, bisphosphonates, antibiotics, antimicrobial agents, cytostatic agents, calcium ATPase and pyrophosphatase pump inhibitors, calcium phosphate-crystal dissolving agents, agents effective-against calcium phosphate-crystal nucleation and crystal growth, and a combination of supportive agents.

54. A transdermal preparation designed to administer a pharmaceutically effective amount of at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents.

55. The transdermal preparation of claim 54, wherein the calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents are present in a concentration sufficient that when applied to the skin a pharmaceutically effective plasma concentration in the patient of said preparation is produced.

56. A transdermal delivery system for application to the skin of a patient, comprising:
(a) a drug impermeable backing layer;
(b) an adhesive layer;
(c) a drug permeable membrane, wherein the membrane is positioned relative to the backing layer so as to form at least one drug reservoir compartment between the membrane and the backing layer; and
(d) a composition comprising at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents are present in a concentration sufficient that the transdermal delivery system has an input rate when applied to the skin sufficient to produce a pharmaceutically effective plasma concentration in the patient.

57. A subcutaneous implant comprising at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents.

58. The subcutaneous implant of claim 57 wherein said implant is effective to release levels of at least one of calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents over an extended period of time when subcutaneously implanted in a human or animal in need thereof.

59. A method of reducing active calcification nidi, comprising administering to a mammal a pharmaceutical composition in an amount that destroys Nanobacteria.

60. The method of claim 59, wherein said Nanobacteria is destroyed by at least one of antibiotics, antimicrobial agents, anti-metabolites, or cytostatic agents.

61. A method of blocking calcium and phosphate accumulation into a vesicle derived from dead host cells, or acidocacisome-like cell organelle, or Nanobacteria or a comparable delineated entity, comprising administering a pharmaceutically effective composition.

62. The method of claim 61, wherein said pharmaceutically effective composition comprises at least one of a calcium ATPase and pyrophosphatase pump inhibitor.

63. The method of claim 62, wherein said ATPase and pyrophosphatase pump inhibitor is selected from at least one of bisphosphonates, vitamin C, vanadate, fluoride, N-ethylmaleimide, N,N-dicyclohexyl carbodiimide, imidodiphosphate, bafilomycin A or calcimycin or antibiotics.

64. A method of reducing and/or preventing calcium phosphate-crystal nucleation and crystal growth, comprising administering a pharmaceutically effective composition.

65. The method of claim 64, wherein said pharmaceutically effective composition comprises at least one of pyrophosphate and its analogs; bisphosphonates; bisphosphonate, tetracycline and other calcium crystal poisons; synthetic, manufactured or naturally occurring protective molecules; Nephrocalcin; Tamm-Horsfall protein; osteopontin; urinary prothrombin fragment 1; bikunin; chondroitin sulfate (CS); heparan sulfate (HS); hyaluronic acid (HA); and synthetic peptides and carbohydrate chains representing fragments therefrom.

66. A method of dissolving calcium phosphate-crystal, comprising administering a pharmaceutically effective composition.

67. The method of claim 66, wherein said pharmaceutically effective composition comprises at least one of calcium chelator, citrate, lactate and/or other organic and inorganic acidic compounds, and bisphosphonates.

68. A method of at least one of dissolving calcification, preventing calcium-mediated mixed bacterial biofilm formation, protecting against blood clotting and thrombosis induced by exposed calcium surface, improving drug penetration and tissue blood flow, preventing tissue destruction and improving tissue remodeling and tissue healing, and controlling inflammation and immune response comprising administering a pharmaceutically effective composition.

69. The method of claim 68, wherein said pharmaceutically effective composition comprises at least one of bile acids, bile acid derivatives, terpenes, organic solvents, anti-lipemic drugs, statins, anti-platelet agents, anti-blood clotting agents, non-steroidal anti-inflammatory drugs, immunomodulators, amino acids, vitamins, antioxidants, anti cell death agents, matrix metalloproteinase inhibitors, enzyme systems, antibiotics, fluoride, bisphosphonates, calcium chelators, citrate compounds and calcium-sequestering acids.

70. The methods of claims 1 and 2, wherein said calcium chelators are administered in a daily dose within a range from 0.1 to 3,000 mg/day.

71. The methods of claims 1 and 2, wherein said calcium chelators are selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof, administered in a daily dose in the range of 10 to 2,000 mg/day.

72. The methods of claims 1 and 2, wherein said calcium chelators are selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof, administered in a daily dose in the range of 100 to 1,500 mg/day.

73. The methods of claims 1 and 2, wherein said antibiotics are administered in a daily dose in the range of 0.01 to 1,000 mg/day.

74. The methods of claims 1 and 2, wherein said antibiotics are selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof, administered in a daily dose in the range of 0.1 to 750 mg/day.

75. The methods of claims 1 and 2, wherein said antibiotics are selected from at least one of beta-lactam antibiotics, aminoglycoside antibiotics, tetracyclines, trimethoprim and sulpha-trimethoprim combinations, nitrofurantoin, and pharmaceutically acceptable salts thereof, and mixtures thereof, administered in a daily dose in the range of 1 to 500 mg/day.

76. The methods of claims 74 and 75, wherein said beta-lactam antibiotics are selected from at least one of penicillin, phenethicillin, ampicillin, aziocillin, bacmpicillin, carbenicillin, cylclacillin, mezlocillin, piperacillin, epicillin, hetacillin, cloxacillin, dicloxacillin, methicillin, nafcillin, oxacillin, and pharmaceutically acceptable salts thereof.

77. The methods of claims 74 and 75, wherein said aminoglycoside antibiotics are selected from at least one of streptomycin, kanamycin, gentamycin, amikacin, neomycin, pardomycin, tobramycin, viomycin, and pharmaceutically acceptable salts thereof.

78. The methods of claims 74 and 75, where said tetracyclines are selected from at least one of tetracycline, chlortetracycline, demeclocycline, doxycycline, methacycline, oxytetracycline, rolitetracycline, minocycline, sancycline and pharmaceutically acceptable salts thereof.
